(19) United States
(12) Patent Application Publication
(10) Pub. No.: US 2005/0119723 A1
(43) Pub. Date: Jun. 2, 2005

(54) Medical device with porous surface containing bioerodable bioactive composites and related methods

(75) Inventor: James Clayton Peacock III, San Carlos, CA (US)

Correspondence Address: PRESTON GATES & ELLIS LLP, 1900 MAIN STREET, SUITE 600, IRVINE, CA, 92614-7319, US

(73) Assignee: Medlogics Device Corporation, Santa Rosa, CA (US)

(21) Appl. No.: 10/724,453

(22) Filed: Nov. 28, 2003

(51) Int. Cl. A61F 2/06

(52) U.S. Cl. 623/1.15; 623/1.38; 623/1.42; 623/1.46; 118/308

(57) ABSTRACT

An implantable medical device includes a porous surface with a composite material located within the pores that includes a bioerodable material in combination with a bioactive agent. The composite material is adapted to erode upon exposure to the body of a patient, thus releasing the bioactive agent into the patient, whereas the porous surface remains on the device. In one embodiment, the composite material includes micro- or nano-particles that are deposited within the pores. In a further embodiment, the porous surface is an electrolessly electrochemically deposited material. Certain tie layer and other surface modification aspects are described to enhance various aspects of the bioactive composite surface. The bioactive composite surface is of particular benefit when provided on an endolumenal stent assembly in a manner adapted to elute anti-restenosis or anti-thrombosis agents or combinations thereof.

### CROSS-REFERENCE TO RELATED APPLICATIONS

Not Applicable

### BACKGROUND OF THE INVENTION

1. Field of the Invention

The present invention relates generally to surfaces of implantable medical devices. More specifically, it relates to depositing a coating incorporating one or more bioactive agents on the surface of an implantable device. Still more particularly, it relates to providing an implant with a substantially porous surface that contains within its pores a composite of a bioerodable material in combination with a bioactive agent.

2. Description of Related Art

Implantable devices include, for example, stents, stent-grafts, embolic filters, detachable coils, pacemaker and defibrillator leads, plates, screws, spinal cages, dental implants, ventricular assist devices, artificial hearts, artificial heart valves, annuloplasty devices, artificial joints, and implantable sensors. Frequently, implanted medical apparatus must be designed to be sufficiently biocompatible to the host body. Otherwise, the body will manifest a rejection of the implant by way of a thrombotic, inflammatory or other deleterious response.

Such implantable devices, therefore, are designed or fabricated from materials possessing surface properties that minimize bodily response at the tissue-device interface. For example, stainless steel is a frequently used implant material due to the relatively passive oxide layer which forms on its surface. Moreover, much activity recently has been directed towards local delivery of bioactive materials into the target tissue via the device being implanted therein. Such bioactive materials are not limited to therapies for treating diseased or abnormal conditions, but also for minimizing the body=s response to both the presence of and injury caused to the tissue during the implantation procedure.

These bioactive materials can include, without limitation, anti-inflammatory agents, anti-infective agents, anti-cancer agents, immunosuppressant agents, and in particular agents used for preventing vascular events or disease such as anti-restenosis compounds and anti-coagulant compounds. With regard to the latter, much research and development has been devoted to one particular implantable medical device for local delivery of bioactive compounds for treating vascular disease, more specifically, stents.

In recent years, intervention in the form of stenting has become widespread in the treatment of peripheral and coronary vascular disease. Stents are mechanical scaffolding devices typically used to maintain the patency of the previously occluded or stenosed vessel following or during percutaneous translumenal angioplasty (PTA), percutaneous translumenal coronary angioplasty (PTCA), or atherectomy or ablation procedures (e.g. directional coronary atherectomy or “DCA” or rotational ablation).

For further illustration, PTA or PTCA typically involves advancing a catheter, having an inflatable balloon on the distal end thereof, through a patient's arterial system until the balloon crosses an atherosclerotic lesion. The balloon is then inflated to dilate the artery. After dilation, the balloon is deflated and the catheter removed leaving an enlarged arterial passageway or lumen, thereby increasing blood flow. Following this procedure, a stent delivery system, which, in the instance of a balloon-expandable stent consists of a stent mounted on a similar balloon catheter or in the instance of a self-expanding stent consists of a stent loaded into the distal end of a delivery catheter, is advanced to the site, expanded and left in-situ to scaffold or prop-up the artery and maintain its patency. Alternatively, in certain procedures, the first step of pre-dilatation may be omitted in favor a direct stenting procedure whereby the stent delivery system dilates at the time of stenting. A significant number of PTA and PTCA procedures, however, result in a restenosis or re-narrowing of the lumen.

Re-narrowing or restenosis of the treated arteries, for example, occurs at a rate of 20% to 50% in patients undergoing this procedure, requiring repeat intervention either, for example, by further stenting, vascular grafting, debulking or bypass surgery. Any one individual's restenosis rate is dependent upon a number of morphological and clinical variables.

In addition to, and with respect to coronary artery intervention, the cellular response from angioplasty or stenting which, besides opening a previously occluded artery, also can cause fissuring of the atherosclerotic plaque and injury to resident vascular smooth muscle cells (“VSMCs”). In response to this injury, among other responses, VSMC hyperplasia, or rapid proliferation of the VSMCs, occurs. Over a period of time, typically between about one to six months, this hyperplastic response can cause significant re-narrowing of the lumenal space opened by the intervention.

Endolumenal interventions of other lumens than coronary arteries also carry within them concerns related to tissue response to injury or device implantation. Such includes for example any other body conduit or lumen that tends to improperly constrict as a result of disease or malfunction, such as: arteries located within the mesentery, peripheral, or cerebral vasculature; veins; gastrointestinal tract; biliary tract; urethra; trachea; hepatic shunts; and fallopian tubes. Moreover, many varied forms of device implants also generally benefit from surface modifications to enhance implant acceptance and/or overall therapy, such as by including desired bioactive agents on, or eluting from, the surface.

With respect to stents in particular, most such implantable scaffolds fall within one of two categories: self-expanding stents and balloon-expandable stents. Self-expanding stents are typically made from nickel-titanium alloys, such as NITINOL, or stainless steel wire or wire braid. Such stents are typically compressed into a first shape and inserted into a sheath or cartridge positioned at the distal end of a delivery device. When the stent is positioned across the lesion, the sheath is withdrawn causing the stent to radially expand and abut the vessel wall. Balloon-expandable stents are typically introduced into a lumen on a catheter having an inflatable balloon on the distal end thereof. When the stent is at the desired location in the lumen, the balloon is inflated to circumferentially expand the stent. The balloon is then deflated and the catheter is withdrawn, leaving the circumferentially expanded stent in the lumen, usually as a permanent prosthesis for helping to hold the lumen open.

Attempts, both mechanical and pharmacological, to address restenosis include providing a suitable surface within the lumen for more controlled healing to occur in addition to the support provided by a stent. Mechanical attempts include providing a lining or covering in conjunction with a stent, such as a stent-graft. The covering of a stent-graft may prevent excessive tissue prolapse or protrusion of tissue growth through the interstices of the stent while allowing limited tissue in-growth to occur to enhance the implantation. The surface of the graft material at the same time prevents scarring from occluding the lumen and minimizes the contact between the fissured plaque and the hematological elements in the bloodstream. Both self-expanding and balloon-expandable stents can be used in conjunction with a covering or lining.

Pharmacological attempts have involved systemic delivery of drugs either orally, intravascularly or intramuscularly. More recently, drug eluting stents have been developed and under commercial use in order to enhance the result of endolumenal stenting. These drug eluting stents typically involve a balloon-expandable stent modified to deliver anti-thrombotic or anti-restenotic compounds. Such devices typically involve the application of a coating to the surface of the stent, specifically adapted to hold and release drugs. Most such coatings are polymers that perform such a hold-and-release function. These polymers can be biodegradable, wherein the coating releases the drug via degradation of the polymer, or non-biodegradable, whereby the drug diffuses therefrom into the surrounding environment.

These polymeric coatings, however, have certain limitations and shortcomings. In one regard, the degradation kinetics of polymers is often unpredictable. Consequently, it is difficult to predict how quickly a bioactive agent in a polymeric medium will be released. If a drug releases too quickly or too slowly from the polymeric medium, the intended therapeutic effect may not be achieved. In another aspect, in many instances, polymeric materials produce an inflammatory response. For example, certain polymeric coatings on stents have been observed to produce an inflammatory response, exacerbating restenosis.

Moreover, it is generally very difficult to achieve robust adhesion between such polymer coatings with the substantially different substrate of the stent scaffold, typically a very different metal substrate. Such challenge may result in delamination, cracking, or other adverse results to the shortcomings of material interface in the intended “composites”. Repeatability, uniformity, and adhesion are difficult to achieve in manufacturing, which may result in poor yields and thus increased costs of products, and may result in adverse consequences after implantation. Mismatched properties such as different thermal and/or mechanical properties between the polymeric coating and the underlying substrate contribute to this difficulty. Inadequate bonding or adhesion between a stent and an overlying polymeric coating may result in separation of these components over time, an undesirable characteristic for an implanted medical device to exhibit. Such separation is even more susceptible at areas of the stent subject to greater amounts of deflection during expansion, such as the apices or crowns of the stent.

Yet another limitation is that it is difficult to evenly coat complex geometries and small objects not to mention small, complex metallic objects with a polymeric material. Therefore, small metallic objects, such as stents, become more difficult to coat evenly with a polymeric material. Yet a further limitation of polymer coatings is that they contribute bulk but do not contribute to the function of a stent which is to maintain lumen patency.

One proposed alternative to polymer coating is sintering. In such a sintering process, a heat and/or pressure treatment is used to weld small particles of metal to the surface of the structure. A porous metallic structure is created. Such sintered metallic structures, however, exhibit relatively large pores. When a bioactive material is loaded into the pores of a sintered metallic structure, the larger pore size can cause the biologically active material to release too quickly, possibly during delivery to the intended tissue. Also, because a high temperature is used to form a sintered structure, a bioactive material must be loaded into the sintered structure after the porous structure is formed. This method is not only time consuming, it is also difficult to impregnate the pores of the sintered structure with the biologically active material. Consequently, it is difficult to fully load the sintered structure with the bioactive material. Nevertheless, even sintering may provide beneficial results as a non-polymeric surface that may present robust adhesion integrity within the underlying metal stent substrate.

More recently, drug delivery from electrochemically deposited thin metal films has been posited. This coating process employs, as one of its steps, an electroless metal deposition. The drug to be delivered is dissolved or dispersed into a metalizing deposition bath, and is co-deposited on the implantable device. This deposition process involves the use of some heavy metals, such as stannous and palladium, to sensitize and activate the surface, which, albeit in small amounts, remains on the implantable device.

Various efforts have been previously disclosed for achieving drug delivery from medical device surfaces by incorporating drugs or other bioactive agents into bioerodable or biodegradable materials, such as for example polymers, on the medical device surface. Certain such disclosures have included coating endolumenal stents with such composite materials. Additional specific disclosures have intended to construct such devices, including for example stents, from the combination drug-bioerodable matrix. Various material and design considerations are inherent in such applications, including in one regard providing the composite matrix with sufficient polymer component in the drug:polymer ratio to exhibit necessary integrity as a coating or mechanical scaffold. However, the biocompatibility of the polymer component is yet to be well settled and generally subject to scrutiny by leading molecular biologists and pathologists. This particularly applies implants where the polymer components are released into vessel walls. Inflammation and foreign body reactions remain a concern principally due to the level of polymer burden within the erodable composite.

Notwithstanding the foregoing, much progress has been postulated regarding developing such bioerodable carriers for drug delivery composites for improved biocompatibility results. Nonetheless, there would be much benefit to a process which provides for bioerodable drug carrier vehicles with drug:polymer ratio having relatively low polymer component and higher drug component.

Further detailed examples of small particle drug carrier vehicles for use in medical applications, or related to bioerodable drug carrier vehicles, or otherwise related to drug delivery vehicles for local therapy of restenosis or other tissue conditions are variously disclosed in the following issued U.S. Pat. No.: 5,271,961 to Mathiowitz et al.; U.S. Pat. No. 5,275,820 to Chang; U.S. Pat. No. 5,562,099 to Cohen et al.; U.S. Pat. No. 5,639,441 to Sievers et al; U.S. Pat. No. 5,674,495 to Bowersock et al.; U.S. Pat. No. 5,733,925 to Kunz et al.; U.S. Pat. No. 5,741,478 to Osborne et al.; U.S. Pat. No. 5,770,609 to Grainger et al; U.S. Pat. No. 5,833,891 to Subramaniam et al.; U.S. Pat. No. 5,869,103 to Yeh et al.; U.S. Pat. No. 5,874,029 to Subramaniam et al; U.S. Pat. No. 5,922,340 to Berde et al.; U.S. Pat. No. 5,942,241 to Chasin et al.; U.S. Pat. No. 5,981,568 to Kunz et al.; U.S. Pat. No. 5,933,805 to Sutton et al.; U.S. Pat. No. 6,017,310 to Johnson et al.; U.S. Pat. No. 6,046,187 to Berde et al.; U.S. Pat. No. 6,068,600 to Johnson et al.; U.S. Pat. No. 6,071,497 to Steiner et al.; U.S. Pat. No. 6,074,659 to Kunz et al; U.S. Pat. No. 6,095,134 to Sievers et al.; U.S. Pat. No. 6,117,449 to See et al.; U.S. Pat. No. 6,129,761 to Hubbell; U.S. Pat. No. 6,171,609 to Kunz; U.S. Pat. No. 6,197,789 to Grainger et al.; U.S. Pat. No. 6,207,185 to See et al.; U.S. Pat. No. 6,221,977 to Park et al; U.S. Pat. No. 6,224,794 to Amsden et al.; U.S. Pat. No. 6,238,702 to Berde et al.; U.S. Pat. No. 6,248,345 to Goldenheim et al.; U.S. Pat. No. 6,248,720 to Mathiowitz et al.; U.S. Pat. No. 6,262,034 to Mathiowitz et al.; U.S. Pat. No. 6,262,079 to Grainger et al.; U.S. Pat. No. 6,358,989 to Kunz et al.; U.S. Pat. No. 6,403,672 to Randolph et al.; U.S. Pat. No. 6,426,339 to Berde et al.; U.S. Pat. No. 6,428,771 to Steiner et al.; U.S. Pat. No. 6,455,526 to Kohn et al.; U.S. Pat. No. 6,475,779 to Mathiowitz et al.; U.S. Pat. No. 6,491,938 to Kunz et al.; U.S. Pat. No. 6,506,411 to Hunter et al.; U.S. Pat. No. 6,514,193 to Kaplan et al.; U.S. Pat. No. 6,515,009 to Kunz et al.; U.S. Pat. No. 6,521,259 to Chasin et al.; U.S. Pat. No. 6,521,431 to Kiser et al.; U.S. Pat. No. 6,534,081 to Goldenheim et al.; U.S. Pat. No. 6,569,441 to Kunz et al.; U.S. Pat. No. 6,599,928 to Kunz et al.; U.S. Pat. No. 6,620,617 to Mathiowitz et al.; U.S. Pat. No. 6,623,722 to Osborne et al.; U.S. Pat. No. 6,630,121 to Sievers et al.; and U.S. Pat. No. 6,632,457 to Sawhney.

At least one additional example is disclosed in the following U.S. Patent Application Publication: U.S. 2002/0155146 to Mathiowitz et al.

Further examples are disclosed variously among the following PCT International Patent Application Publications: WO 91/06286 to Mathiowitz et al; WO 00/09088 to Sawhney; and WO 01/68745 to Mathiowitz et al.

The disclosures of all the U.S. Patents, U.S. Patent Application Publications, and PCT International Patent Application Publications noted above are herein incorporated by reference thereto to the extent consistent with the various objects and related aspects, modes, embodiments, variations, and features provided herein according to one of ordinary skill based upon review of this disclosure in combination with the incorporated reference.

Drug delivery carrier vehicles such as coatings that incorporate drug into porous surfaces, e.g. porous polymers, also deliver drug via a relatively uncontrolled diffusion gradient modality. Use of multiple layers of varying porosity or permeability to the drug elution have thus been disclosed in order to modify such elution characteristic to a desired time-based elution profile. There would be much benefit to provide the captured drug vehicle within such porous coatings in a manner that modifies the elution characteristic from a simple diffusion gradient.

It is further noted that electroless lectrochemical coating methods for co-deposition of additive materials have been principally used in the past with particulate matter as the co-deposited material. Examples of such particles include for example polytetrafluoroethylene (“PTFE”, such as Teflon™) and diamond chips. Insoluble oil droplets have also been disclosed for co-deposition in an electroless electrochemical coating process. Whereas coating of dissolved or suspended drug molecules (e.g. generally pure powder form of drug as additive to the electrochemical bath) into electroless electrochemical matrices have been achieved, and elution therefrom has been demonstrated, the process of electroless electrochemical co-deposition is considered particularly well-suited for use with preparations of the drug in micro- or nano-particulate form. Thus, such a modified process and resulting structure incorporating particulate drug vehicles within electrolessly electrochemically co-deposited nano-porous structures is considered to provide substantial benefit.

Such drug-carrying particulate co-deposition may be beneficially particles formed of principally only the drug itself. However, depending upon the particular drug and particular electroless electrochemical bath employed for co-deposition, such particles may not maintain integrity during the coating process, or may dissolve, degrading the benefits of providing the particulates within the bath in the first place to enhance co-deposition. Moreover, co-deposition of solely drug particulates within an electrolessly electrochemically formed nano-porous composite matrix is generally expected to elute according to a diffusion gradient profile. There would be additional benefit therefore if such particulate additive were complexed in a composite of drug and another structural material, such as in particular a bioerodable material that suitably maintains the particulate integrity through the co-deposition process and provides suitable elution characteristics in vivo.

### BRIEF SUMMARY OF THE INVENTION

The present invention according to various aspects therefore provides a medical device and related material deposition process that provides a porous outer surface that contains within its pores a bioactive composite with a bioactive agent combined with a bioerodable or biodegradable material. Biodegradation or erosion of the material releases the bioactive agent from the porous outer surface, with the porous outer surface typically left remaining on the medical device.

Accordingly, one particular aspect of the invention is a medical device with a substrate and a porous outer surface on the substrate that includes a plurality of pores. The medical device is adapted to be positioned at least in part at a location such that the pores are exposed to a body of a patient. A bioerodable or biodegradable material is located within the pores in combination with a bioactive agent. At the location, the material is adapted to bioerode or biodegrade such that the bioactive agent is released, and further such that the porous outer surface is left remaining on the substrate.

According to one mode of this aspect, the medical device comprises a long-term implant.

According to another mode, the medical device comprises an endolumenal stent.

According to another mode, the medical device comprises an embolic filter.

According to another mode, the medical device comprises an orthopedic implant.

According to another mode, the medical device comprises a surgical staple.

According to another mode, the medical device comprises a guidewire.

According to another mode, the medical device comprises an electrode.

According to another mode, the medical device comprises an anchor.

According to another mode, the pores are exposed substantially only along the porous outer surface and do not communicate through the substrate.

According to another mode, the pores are less than about 10 microns in diameter.

According to another mode, the pores are less than about 5 microns in diameter.

According to another mode, the pores are less than about 2 microns in diameter.

According to another mode, the pores comprise nano-pores that are less than about 1 micron in diameter.

According to another mode, the pores are inherent in the material and are not post-processed into the material.

According to another mode, the bioerodable material and bioactive agent are combined in the form of discrete particles located within the pores.

According to one embodiment of this mode, the particles are less than about 10 microns in diameter.

According to another embodiment, the particles are less than about 5 microns in diameter.

According to another embodiment, the particles are less than about 2 microns in diameter.

According to another embodiment, the particles comprise nano-particles and are less than about 1 micron in diameter.

According to another embodiment, the ratio of bioactive agent to bioerodable material is at least about 40% percent by weight, and in further embodiments may be at least about 50% by weight.

According to another mode, the bioerodable material comprises a polymer.

According to another mode, the bioactive agent comprises an anti-restenosis agent.

According to another mode, the bioactive agent comprises an anti-thrombin agent.

According to another mode, the bioactive agent comprises an anti-platelet aggregation agent.

According to another mode, the bioactive agent comprises an anti-proliferative agent.

According to another mode, the bioactive agent comprises a growth factor.

According to another mode, the bioactive agent comprises an anti-inflammatory agent.

According to another mode, the bioactive agent comprises an immunosuppressant agent.

According to another mode, the bioactive agent comprises first and second agents, each having a different bioactivity providing for a beneficial combination result.

According to certain further modes, the bioactive agent comprises at least one of: sirolimus; everolimus; tacrolimus; paclitaxel; dexamethasone; nitric oxide; exochelin; des-aspartate angiotensin 1 (DAA-1); steroid (e.g. estradiol); sialokinin; gamma tocopherol; pleiotrophin; VEG-F; IIb/IIIa inhibitor; clopidogrel; heparin; aspirin; combinations or blends thereof; or precursors, donors, or analogs thereof.

Another aspect of the invention is an implantable endolumenal stent comprising a scaffold with a porous outer surface having a plurality of pores. The endolumenal stent is adapted to be implanted at a location within a lumen of a body of a patient such that the scaffold engages a luminal wall that defines the lumen and such that the porous outer surface is exposed to at least one of material flowing within the lumen or the luminal wall. A bioerodable or biodegradable material is located within the pores in combination with a bioactive agent. At the location, the material is adapted to bioerode or biodegrade such that the bioactive agent is released from the porous outer surface, and such that the porous outer surface is left remaining on the scaffold.

According to one further mode, the scaffold comprises a metal.

According to one embodiment of this mode, the metal comprises a cobalt-chromium alloy.

According to one further feature adapted for use according to this embodiment, the porous outer surface comprises a porous composite matrix of cobalt plus a reducing agent of cobalt.

According to another embodiment, the metal comprises a nickel-titanium alloy.

According to one further feature adapted for use according to this embodiment, the porous outer surface comprises a porous composite matrix of nickel plus a reducing agent of nickel.

According to another embodiment, the metal comprises a stainless steel alloy.

According to one further feature adapted for use according to this embodiment, the porous outer surface comprises a porous composite matrix of nickel plus a reducing agent of nickel.

Further aspects of the invention, and that combine in further regards with the various aspects noted above to provide further modes thereof, comprise a thin metal coating and coating process for coating implantable medical devices. In addition, certain modes thereof provide a relatively passive, or relatively non-reactive, external surface coating on implantable medical devices following release of bioactive agents therefrom, lessening the reaction to the device and improving the device-tissue interface, such as for long-term implants. Further such aspects are provided as follows.

Another aspect of the invention provides a medical device with an electrolessly electrochemically deposited porous outer surface that is adapted to exhibit substantially robust adherence to the underlying implantable device substrate so as to provide substantial surface integrity through delivery and in-vivo use of the medical device, and that is adapted to carry and elute substantial quantity of bioactive agent from within the pores.

Another aspect of the invention is a coating preparation that comprises an electroless electrochemical bath with particles suspended therein that include a bioactive agent in combination with a bioerodable or biodegradable material. The bath is adapted to electrolessly electrochemically deposit a metal composite matrix onto a sufficiently activated surface, and also to co-deposit the particles within pores formed within the metal composite matrix.

Another aspect of the invention incorporates one or more therapeutic agents into a surface coating of a medical device.

Another aspect of the invention is a method that coats an implantable medical device without significantly increasing its bulk.

Another aspect of the invention is an implantable medical device with a substrate that is fabricated from a metal or metal alloy, and that is coated with a coating exhibiting material properties that are substantially compatible with the underlying substrate.

Another aspect of the invention provides an improved coating on the surface of an implantable endolumenal prosthesis for maintaining lumen patency.

A further aspect of the invention provides a relatively passive coating encasing a stent having a substrate material that is generally more bioreactive than the coating.

A further aspect of the invention provides an improved thin metal coating process for deposition onto implantable endolumenal devices. A further mode of this aspect co-deposits therapeutic agents with and within the coating for subsequent elution from the implantable medical device.

The invention according to still further aspects comprises a method of forming multiple layers on the surface of a device to form a composite matrix. In a particular embodiment, a first layer is applied or struck on the surface by contacting the surface with an electrolytic solution containing metal ions, and followed by subsequently electrodepositing a thin metal film onto the surface. This is followed by contacting the surface with a second electrochemical bath containing metal ions and one or more therapeutic agents to form a second layer on the surface of the device. The agents are co-deposited with the metal ions on the surface of the device to form a composite, bioactive, metallic matrix on the device.

In a further mode, the first layer is electroplated onto the surface of the device and the second layer is deposited through an electroless electrochemical co-deposition process.

The invention according to still further aspects includes the application of one or more electroplated layers, and one or more layers deposited through an electroless electrochemical process. In a further embodiment, the electroless electrochemical deposition steps are performed without any pre-sensitizing of the surface nor any pre-deposition of a catalyst on the surface to be coated.

Further objects and advantages of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawing which is for illustrative purposes only:

FIG. 1 shows a schematic longitudinal cross-sectioned view through a substrate coated according to one embodiment of the invention.

FIG. 2 shows a further exploded longitudinally cross-sectioned schematic view of finer detail of a porous bioactive coating useful according to the embodiment shown in the more general view of FIG. 1.

FIG. 3 shows a longitudinally cross-sectioned view of another bioactive coated substrate surface according to a further embodiment of the invention.

FIG. 4 shows a longitudinally cross-sectioned view of another bioactive coated substrate surface according to a further embodiment of the invention.

FIG. 5 shows a schematic flow diagram of one method embodiment of the invention.

FIG. 6 shows a schematic flow diagram of another method embodiment of the invention.

FIG. 7 shows a cross-sectioned schematic view of an illustrative coating environment adapted for use according to various of the embodiments of the other FIGS.

FIG. 8 shows a schematic partially longitudinally cross-sectioned view of a stented lumen such as a coronary or peripheral arterial vessel.

FIG. 9 shows a schematic transversely cross-sectioned view through an illustrative strut of a stent adapted for use according to the mode shown in FIG. 8, and further with respect to various of the embodiments shown in the prior FIGS.

### DETAILED DESCRIPTION OF THE INVENTION

It is to be appreciated therefore that the following detailed description of various embodiments are illustrative of devices and methods that generally achieve one or more objects, aspects, modes, embodiments, variations or features of the invention. The following description is therefore illustrative and, though providing highly beneficial modes and embodiments intended to be given independently patentable weight, is nonetheless not intended to limit the various broad aspects of the invention which are to be accorded the widest scope consistent with the principles and features described herein. Various modifications to the preferred embodiment will be readily apparent to those skilled in the art and the generic principles herein may be applied to other embodiments.

Therefore, certain highly beneficial aspects of the invention are thus illustrated according to the detailed description and by reference to the accompanying figures as follows.

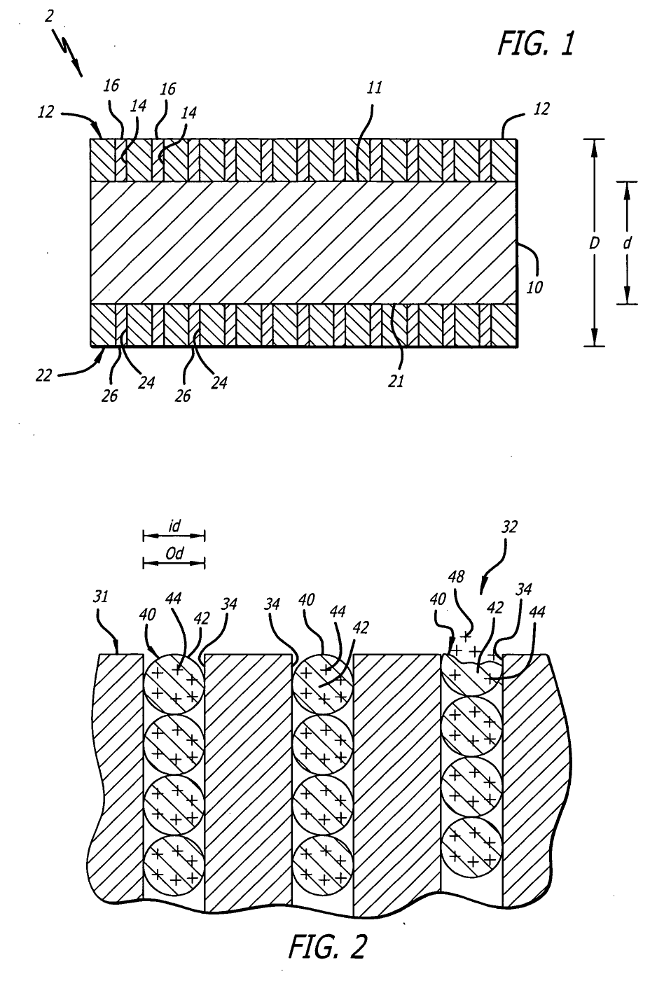

FIG. 1 shows a schematic view of a coated medical device implant 2 that illustrates certain aspects of the invention as follows. A substrate 10 is coated on a first surface 11 with a composite coating that includes a porous coating 12 with a plurality of pores 14 that are filled with a composite material 16. Composite material 16 includes a bioerodable material in combination with a bioactive agent. A second surface 21 is also coated similarly, with a composite coating having a porous coating material 22 with a plurality of pores 24 filled with bioerodable composite material 26 similar to that described for material 16. First and second surfaces 11,21 illustrate for example a luminal wall and inner luminal surface of a stent, respectively.

Again, the various features shown and described are illustrative, and relative sizes and orientations, etc., of such components may be modified to suit a particular need, and in general are exaggerated for clarity, such as for example pores 14 which in particular beneficial embodiments are very small, e.g. micropores, and in further beneficial embodiments sub-micron sized nano-pores.

In various embodiments related to coronary stents for example, such sized pores will typically have a diameter of up to about 10 microns, most typically about 5 microns, and in highly beneficial embodiments less than about 2 microns and even less than about 1 micron as nano-pores. Such porosity would be typically located on an underlying stent substrate having a diameter d of at least about 25 microns, generally up to about 55 microns, and whereas the resulting coated composite implant 2 would typically have illustrative cross-sectional diameters D between about 25 microns to about 75 microns, and generally between about 30 microns to about 50 microns. Coating thicknesses, (D minus d)/2, will typically range between about 2 microns to about 20 microns, and more typically between about 2 microns and about 10 microns. Again, all such dimensions are provided for further illustration of certain particular further embodiments, and other specific dimensions of the component parts, or differing relative comparative dimensions between them, are contemplated as would be apparent to one of ordinary skill.

FIG. 2 shows cross-sectional detail under a more exploded higher magnification schematic view of the porous composite coating similar to that adapted for use according to the FIG. 1 embodiment. More specifically, composite coating 32 includes a porous coating 31 that includes a plurality of pores 34 that each contains a plurality of composite particles 40. These composite particles 40 include a carrier material 42 in combination with a bioactive agent 44 according to certain broad aspects illustrated by the present embodiment. More specific to the particular embodiment shown, however, material 42 is a bioerodable material, which when exposed to the biological environment within a patient erodes to release the bioactive agent 48 into the surrounding environs. This is shown for illustration on the right side of FIG. 2.

Further to this particular embodiment, the particles 40 are shown with an outer diameter od that generally matches the inner diameter id of the pores 34. This is achieved for example by depositing these materials together, such as in an electroless electrochemical co-deposition process, wherein the formation of pores 34 may be self-defining around the particles 40 being co-deposited. Other methods may also be employed, such as for example by post-processing a pre-formed porous coating 31 with a later step depositing particles 40, which may self-differentiate to particles equal to or less than the inner diameter id of the pores 34, keeping larger particles out. In this regard, even the co-deposition process of the electroless electrochemical embodiments may include differing relationships between pore sizes and the sizes of the particles deposited. Again, as noted above for FIG. 1, the relative sizes and conformations of the pores is provided in a particular relationship for clarity of illustration, and may vary depending upon the particular methods used or desired results.

The composite coatings just described by reference to FIGS. 1 and 2 were highly schematic and illustrative of a wide variety of particular coating schemes and resulting structures. Further specific embodiments follow for further illustration.

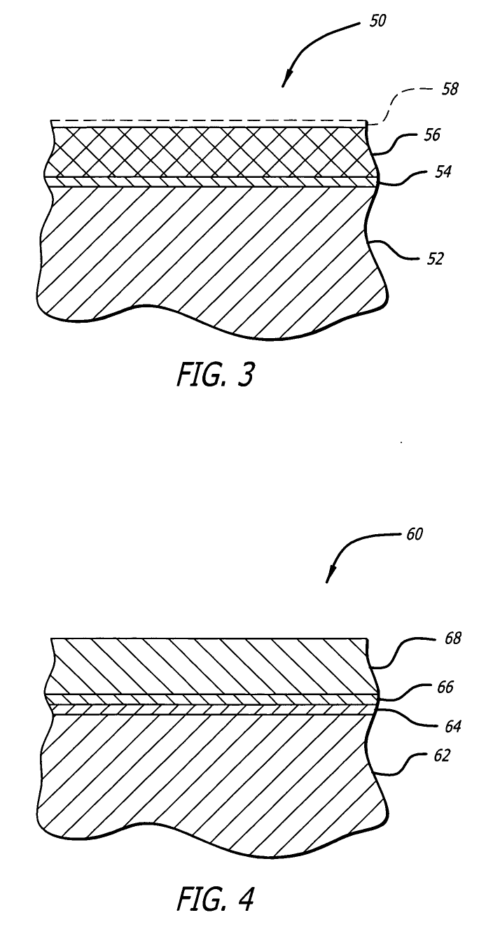

FIG. 3 shows a schematic cross-sectioned view of a further composite coating embodiment related to a coated medical device implant 50 as follows. Here, a substrate 52 is coated with a porous composite coating layer 56 which may be similar for example to that shown and described above by reference to FIGS. 1 and 2. In addition however, an additional coating layer 54 is shown located between the composite drug-loaded composite layer 56 and the underlying substrate 52. This additional layer 54 may be for example a tie layer in order to achieve optimal adhesion of the surface composite through the intended environment of use. In one particular further embodiment for example, the layer 54 may be an electroplated layer of nickel deposited for example onto a nickel-containing substrate 52 such as stainless steel or nickel-titanium, and overcoated by a composite drug coating layer of nickel-phosphorous providing micro- or nano-pores containing drug or drug-bioerodable composite material. This particular relationship of layers and corresponding materials is considered highly beneficial for example according to further detailed embodiments described in further detail below, and in particular beneficial relation to stents.

Accordingly, various different schemes of layered coating materials may be employed and remain consistent with the various broad objects and aspects of the present invention.

In a further example to illustrate, an outer layer 58 (shown in shadow in FIG. 3), may also be added over the porous composite coating layer 56 provided according the invention. This outer layer 58 may be for example another porous layer of material that modifies the elution of drugs from layer 56, or may be a second drug-carrying layer with different material composition or dosing scheme from that in layer 56. In one particular example, layer 58 carries a different drug agent than layer 56. In another example, it carries a different drug agent dose than layer 56, either of the same bioactive agent, or of a different agent. Such outer layer 58 may also be bioerodable, either as a composite with drug ag nt or as a protective covering that, once erodes, then initiates release of drug from layer 56 with reduced encumbrance from the outer layer 58. This later scheme may, for example, protect the drug coated device during delivery to the target lumen or body space.

Or, a high-drug content bolus of elution may be related to the erosion of outer layer 58 as a bioerodable drug-carrying layer (either similar to layer 56 in general component parts or of other bioerodable drug-carrying vehicles available to one of ordinary skill), whereas a slower elution is related to erosion of the bioerodable contents of layer 56. Such scheme is consistent for example with the desired elution profiles of various anti-restenosis drugs where an initial, relatively high “bolus” of drug is given within the first 24-48 hours of stent implant, followed by slower elution over the ensuing period, e.g. between about 14 to about 28 days. For example, between about 20% to about 80%, and generally between about 20% and about 50%, of all drug may be delivered in the initial period, followed by the substantial remaining drug elution from the device over the subsequent period.

In another multi-layered composite coating embodiment shown in FIG. 4, a coated device implant 60 includes a substrate 62 coated with a composite coating layer 68 that elutes drugs, but providing two tie layers 64,66 between substrate 62 and outer layer 68. These two tie layers 64,66 may be, for example, a first layer 64 that is electroplated metal, followed by the second layer 66 that is an electrolessly electrochemically deposited layer of that metal in combination with a reducing agent of that metal. In specific highly beneficial embodiments for further exemplary illustration, substrate 62 is a nickel-containing metal such as stainless steel or nickel-titanium alloy, layer 64 is electroplated nickel, layer 66 is electrolessly electrochemically deposited nickel-phosphorous composite matrix, and layer 68 is electrolessly electrochemically deposited nickel-phosphorous composite matrix that is substantially porous and contains a bioactive agent, such as in a bioerodable composite matrix or particles as shown and described by reference to FIGS. 1 and 2 above. Moreover, an additional outer coat similar to layer 58 may also be added consistent with the objects and aspects described above for that layer by reference to FIG. 3.

It is to be further appreciated that various methods may be employed to produce the intended result according to certain aspects of the invention that includes a bioerodable drug composite matrix within pores of a porous surface on a device implant.

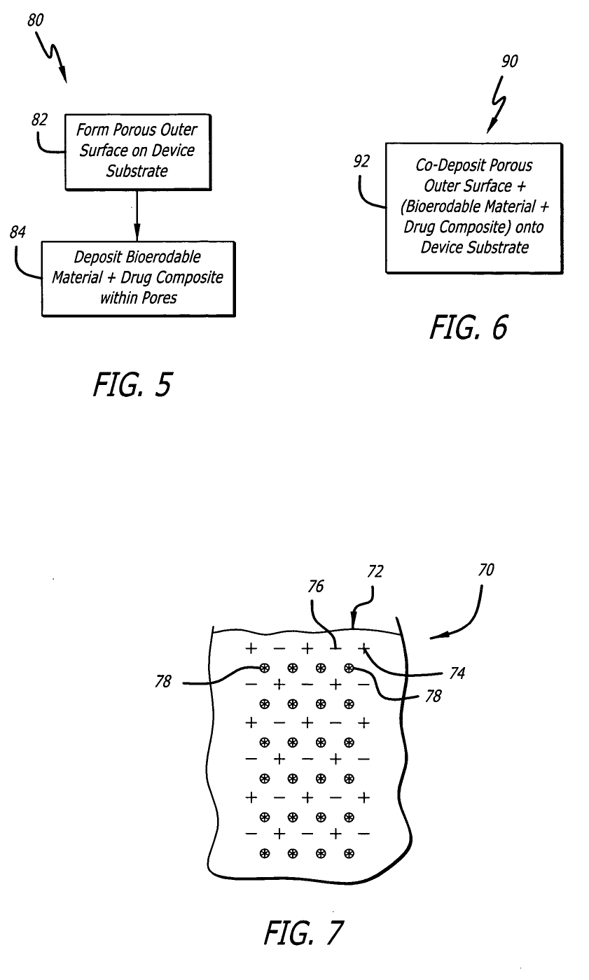

One particular example is shown in FIG. 5, wherein the coating method 80 includes a step 82 that forms a porous outer surface on a device substrate, followed by a step 84 that deposits a composite material with a bioerodable material in combination with a bioactive agent or drug within the pores of the porous outer surface first formed.

Such exemplary method may include various different porous surface treatments or coatings, e.g. polymers, sintered materials such as metal, ceramics, etc. Various different compositions and methods for depositing the bioerodable composite matrix into the pores are also contemplated, as would be apparent to one of ordinary skill based upon review of this disclosure and other available information. Such includes for example exposing the porous coating to solvent solutions that cure, enlisting the aide of elevated pressures to deposit within the pores, fluid baths, atomized or nebulized environments of the depositing matrix material and/or its component parts, and including various particular preparations of micro- or nano-particles, etc.

In another highly beneficial mode shown schematically in FIG. 6, a method 90 may be used which simplifies such to lesser steps, e.g. a co-deposition step 92 wherein a porous outer surface is co-deposited onto the desired device substrate together with a composite matrix of bioerodable material in combination with a bioactive agent or drug.

Such latter method 90 described by reference to FIG. 6 is in particular a highly beneficial mode resulting from the use of electroless electrochemical co-deposition methods. Such methods and resulting materials and surface treatments are variously noted above for illustration purposes to the previous embodiments, and further described in more detail according to certain particular beneficial embodiments below.

Electrolessly Electrochemically Co-Deposited Nano-Porous Drug-Elution Coating

As mentioned by reference to the embodiments of FIGS. 1-4 above, various modes of the invention are well suited for use with electrolessly electrochemically deposited porous composite coating matrix. This may be achieved according to a number of specific electroless electrochemical coating formulations and methods, with a variety of specific composite coating results.

A schematic view of a typical electroless electrochemical deposition bath is shown in FIG. 7 for illustration purposes. More specifically, an electroless electrochemical bath 72 is provided within a coating environment or container (e.g. suitable beaker, etc.), and includes among other component ingredients a soluble volume of metal ions 74 in a particular ratio combination with opposite valence ions or salts 76 that function as a reducing agent of the metal ions. Further included in the bath 72 is a suspended volume of micro- or nano-particles 78 that are composite materials of bioerodable material in combination with a bioactive agent. As elsewhere described herein, by exposing the properly active substrate surface to this bath formulation, the respective metal and reducing agent together form a composite matrix onto the exposed and active surface that captures the bioerodable drug composite particles within pores formed around those particles.

The result is a composite coating with the porous electrolessly electrochemically deposited metal-reducing agent composite and with the bioerodable-drug composite particles within those pores. A simple drying step after removal of the substrate (or otherwise removal of the catalytic ingredients such as under forced air or other inert gas) is often all that is required for a final result.

The foregoing description is highly beneficial mode to provide a desired bioactive device implant surface, such as consistent with the various embodiments described above with respect to FIGS. 1-4. However, further detail is provided below with respect to more particular beneficial coating modalities, and is to be appreciated in combination with these present embodiments among FIGS. 1-4, as well as 5-7 (to the extent modified to provide drug loading separate from the porous coating formation with respect to FIG. 5). It is also to be appreciated that such further detailed embodiments with respect to specific coating methods and formulations provided below have further reaching benefits, other than in combination with the embodiments noted above.

Accordingly, a thin metal-based coating and a process depositing the thin metal-based coating on implantable endolumenal medical devices is provided according to further aspects of the invention as follows.

In one particular mode, an improved method is provided for depositing a thin metal matrix onto the surface of an implantable device. The multiple step process deposits a composite thin metal matrix onto the device's surface. This multiple step process also includes one or more steps where a therapeutic or biologically active agent, or agents, is co-deposited with and within one or more thin metal films. The process is quite controllable, including with controlled variability of results, based on adjusting such parameters as temperature, pH, relative concentration of solution constituents, other additives or agents present in solution and time.

Specifically, the present invention makes use of the process of electroless electrochemical deposition to apply one or more layers of thin metal film, incorporating one or more biologically active agents, onto the surface of an implantable device. According to one distinct benefit, electroless electrochemical deposition generally progresses as a self-assembling, autocatalytic process.

More specifically, in a further embodiment, the process of electroplating a surface is combined with electroless electrochemical deposition, in a multi-step approach. In one particular regard, such method has been observed to provide better adherence of the metallic matrix to the surface of the underlying device while also allowing for the incorporation of one or more biologically active agents with and within the coating matrix.

By one further more particular embodiment therefore, two solutions are prepared. The first solution is an electroplating or electrolytic solution or bath. The second solution is an electroless deposition solution or bath. The first bath is formed with a cathode (the device to be coated), and an electrolytic solution containing metal ions. The second bath is formed using metal salts, a solvent solution (e.g. aqueous environment), a reducing agent, and one or more biologically active agents to be incorporated into the coating matrix. Other materials are typically included in such second electroless electrochemical bath, as has been previously described and available to one of ordinary skill.

Prior to subjecting the device to the electrochemical processes described, the surface of the device is typically pre-treated in order to be appropriately prepared for suitable activity allowing for deposition thereon. Often, this aspect of the method includes de-oxidation of the surface, such as in the case of using substrate alloys such as stainless steel, cobalt-chromium, or nickel-titanium alloys that rapidly form generally non-reactive oxide layers on their surfaces exposed to oxygen rich environments. This may be accomplished for example by contacting or immersing the device in a pretreatment bath, which may include for example organic or inorganic acids. For example, with regard to alloys such as stainless steel, nickel-titanium, or cobalt-chromium an acid bath (or series thereof) may be used that includes one or a combination of inorganic acids such as hydrochloric acid (HCl), nitric acid (HNO₃, or hydrofluoric acid (HF). Other methods of cleaning the surface can include molten salts, mechanical removal, alkaline cleaning, or any other suitable method that provides a clean, coatable surface. This initial step generally serves to clean the surface and etch the surface thereby removing any resident oxide layers on the structure and pitting the surface to improve subsequent adherence of the coating to the device.

The device is then rinsed, preferably deionized water and more preferably, deionized and distilled water. Although, other suitable liquids or gasses could be used to remove any possible impurities from the surface. After rinsing, the implantable structure to be coated is immersed in the first bath. A current is then applied across the device causing the metal ions to move to the device and plate the surface. This electroplating step causes an intermediate or “strike” layer to be formed on the surface of the device. Metal ions for this first bath are typically chosen to be compatible with the material making up the device itself. For example, if the underlying structure is made of cobalt chrome, cobalt ions are preferred. It has been observed that this strike layer improves overall adherence of the coating to the implantable device as well as increasing the rate of deposition or efficiency of the second, electroless film. The device is subsequently removed from the first bath, and may be rinsed again with water prior to immersion into the second bath.

The device is then immersed in the second, electroless bath at a controlled temperature and pH value. In this step, metal ions, the reducing agent, and the one or more therapeutic agents are simultaneously and substantially uniformly, co-deposited on the struck surface of the device. After immersion in this second bath, a bioactive composite metallic matrix has been formed on the surface of the device. The device is removed from the second bath and allowed to dry.

By this deposition process, any suitable structure can be coated. The device can be porous or solid, flexible or rigid, have a planar or non-planar surface. Accordingly, in some embodiments the device could be stent, a pellet, a pill, a seed, an electrode, a coil, etc. The device to be coated may be formed of any suitable material such as, metal, metal alloy, ceramic, polymer, glass, etc.

Any suitable source of metal ions can be used for the first electrolytic bath. Typically, such metal ions are derived from metal salts which dissociate from one another in solution. Such salts, and therefore ions, are well known in the field of electrolytic deposition and can be chosen by those of ordinary skill in this art. Examples of suitable metal ions depends on the underlying device to be coated, but does include ions of nickel, copper, gold, cobalt, silver, palladium, platinum, etc., and alloys thereof. Different types of salts can be used if it is desired to strike a metal alloy matrix on the surface of the device.

Similarly, any suitable source of metal ions can be used for the second electroless electrochemical deposition bath. Such are also typically derived from metal salts. Examples of such suitable sources depend on the underlying device to be coated and are well known in the field of electroless electrochemical deposition and can be selected by those of ordinary skill in this art.

The electroless electrochemical solution also generally includes a reducing agent and may include complexing agents, buffers and stabilizers. The reducing agent reduces the oxidation state of the metal ions in solution such that the metal ions deposit on the surface of the device as metal. Complexing agents are used to hold the metal in solution. Buffers and stabilizers are used to increase bath life and improve stability of the bath. Buffers are also used to control the pH of the solution. Stabilizers are also used to keep the solution homogeneous. Examples of such complexing agents, buffers and stabilizers are well known in the field of electroless electrochemical deposition and can be selected by those of ordinary skill in this art.

Concerning the therapeutic agents to be co-deposited, any such agent, agents, or combinations thereof can be deposited within the coating depending on the condition to be treated, response desired, or tissue into which the device is to be introduced. Agents which can be coated onto the surface of the device in accordance with the invention include for example the following compounds; organic, inorganic, water soluble, water insoluble, hydrophobic, hydrophilic, lipophilic, large molecules, small molecules, proteins, anti-proliferatives, anti-inflammatory, anti-thrombogenetic, anti-biotic, anti-viral, hormones, growth factors, immunosuppressants, chemotherapeutics, etc.

These therapeutic agents are co-deposited or captured within the electroless electrochemically deposited layer, diffuse out or are released from the coating via pores formed in the coating by the coating process itself. The metal composite matrix forms pores between self-assembling grains as they meet and grow on the surface being coated. This porosity, or the extent and nature of these pores, is a property that is readily manipulated according to proven methods well known to those of ordinary skill in this art.

With regard to the first electroplating bath, in another embodiment of the invention, one or more intermediate layers can be struck on the surface of the device. This can improve the efficiency of the subsequent electroless electrochemical coating step.

Likewise, with regard to the second electroless electrochemical bath, one or more films can be coated onto the surface of the device. Furthermore, multiple electroless electrochemical baths can be used such that not all these baths co-deposit one or more therapeutic agents. For example, after the electroplating step, a first electroless electrochemical bath without any therapeutic agents can be employed to place a first electroless coating onto the surface of the device. The device can then be transferred to a second electroless bath containing one or more therapeutic agents in solution. This can improve the efficiency of the step involving co-deposition of the metal ions, reducing agent and one or more therapeutic agents.

Moreover, multiple electroless baths can be prepared containing and co-depositing different biologically active agents in each coating layer. In addition, an electroless bath, not containing any therapeutic agents, can be applied as a top coat to modify or control the release of therapeutic agents from an inner layer or layers.

The invention will now be described in additional detail by way of working examples of the metallic bioactive matrix deposited on stents. The scope of the present invention, however, is not at all limited by these working examples, though their descriptions are independently considered highly beneficial and useful. Nor is the implantable device limited to a stent, though again such application and resulting composite device is independently beneficial and useful. In particular, the intricate microstructures of stents and ability to uniformly, controllably, and robustly coat such microstructures with high degrees of integrity through stent expansion is considered of particular competitive benefit compared with other alternative coating modalities. Nonetheless, with respect to certain broad aspects contemplated hereunder, these examples are illustrative of a manner in which such aspects of the invention can be practiced.

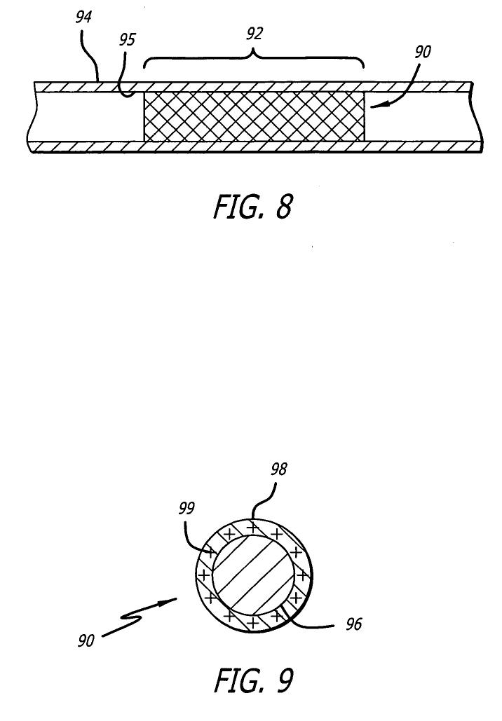

Moreover, with respect to stent applications of the various embodiments herein described, illustrative examples are variously shown in FIGS. 8 and 9. More specifically, FIG. 8 shows a stent 90 implanted along a stenting segment 92 of a lumen 94, such as a coronary or peripheral artery vessel, in an expanded configuration that engages the vessel wall 95 and as a support scaffold holds it open.

A cross-section of an illustrative stent strut is shown in FIG. 9, and includes an underlying scaffold 96 surrounded by an outer coating 98 that includes a bioactive agent 99. As previously described, the scaffold 96 may be of many different specific types of material, but in general typically are metal alloys such as for example stainless steel, nickel-titanium, or cobalt-chrome. With respect to the various coating examples and embodiments herein described, typically beneficial choices for electroless electrochemical coating deposition include nickel-phosphorous composites for the nickel-rich stainless steel and nickel-titanium alloys, whereas a cobalt-phosphorous may be beneficially chosen for the cobalt-chrome alloys. Moreover, the electroplated strike layers for such coatings, if utilized, will often be electroplated nickel for such nickel-rich alloys and nickel-based electroless outer coating composite, or possibly electroplated cobalt or chrome for such cobalt or chrome-containing cobalt-chrome alloys (or with respect to other such applications of cobalt-phosphorous electroless depositions).

### EXAMPLE 1

Bioactive composite coatings were formed on the surface of stainless steel stents. Each stent had two tie layers of nickel struck on its surface prior to immersion in a nickel-phosphorous (Ni—P) electroless deposition bath—further including a de-oxidizing etching stent in-between such multiple nickel strikes. Sirolimus (Rapamycin™), paclitaxel (Taxol™), and des-aspartate angiotensin I (DAA-1) were dissolved/suspended in the various Ni—P baths and co-deposited on the tie layer.

More specifically, each stent was first prepared by immersion in a 37% hydrochloric (HCl) acid bath at room temperature for seven minutes. The stent was then rinsed with de-ionized and distilled water. After rinsing, the stent was immersed in an electrolytic bath containing nickel ions, which bath was concocted by dissolving nickel chloride (NiCl) in HCl and water. The nickel strike was conducted at room temperature. A negative electric charge was then applied to the stent causing the nickel ions to aggregate on the stent surface. A charge of approximately 0.7 volts was applied for about four minutes. Subsequent to this electroplating step, the stent was again rinsed in distilled, deionized water. The stents were again immersed in HCl for seven minutes and again immersed in the strike bath, with charge applied as before, for about four minutes. Following this double strike, the stents were immersed into an electroless Ni—P bath for about ten minutes, which bath included a mixture of NiSO₄, NaH₂PO₂, Na₃C₆H₅O₇, and NH₄Cl to form a homogenous, aqueous solution. The respective amounts of these materials in the bath formulation used were as follows (based upon 1 L preparation in deionized, distilled water): NiSO₄ (30 g/L); NaH₂PO₂ (10.6 g/L); Na₃C₆H₅O₇ (100 g/L); and NH₄Cl (53.6 g/L). Adjustment of the pH was generally performed to target ranges by addition of NaOH.

Rapamycin (sirolimus), paclitaxel, and DAA-1 were also added to various electroless Ni—P baths, of the same composition as the above described bath, and co-deposited therewith on the surface of the stent over the tie layers and initial Ni—P layer. The electroless Ni—P and drug co-depositions were conducted at a temperature range of between about 37-445 degrees C. and a pH of between about 9.5-10 for a total of about 120 minutes each. Paclitaxel and DAA-1 were added to their respective Ni—P baths at a concentration of about 1.25 mg per 25 ml Ni—P solution. Rapamycin was added to its Ni—P bath at a concentration of about 1 mg per 25 ml of Ni—P solution.

Weight measurements were taken before and after coating according to the methods just described. Weight gain is considered a measure of coating deposition. Stents were observed to exhibit marked weight gain after coating deposition with each of the drug baths described.

Drug elution testing in 37 degree aqueous baths was conducted. Elution of compounds into the test baths was confirmed over at least 48 hour time periods from stents coated with each of these drug-loaded baths according to the methods just described.

### EXAMPLE 2

Bioactive composite coatings were formed on the surface of nickel-titanium (Nitinol) self-expanding stents. Each stent had a tie layer of nickel struck on its surface prior to immersion in a Ni—P electroless deposition bath. Rapamycin, DM-1 and sialokinin (“HP-1”) were dissolved/suspended in the various Ni—P baths and co-deposited on the tie layer.

More specifically, each stent was first prepared by immersion in a bath of about 2% hydrofluoric (HF) and about 21% nitric (HNO₃) acid bath at room temperature for about 2 minutes. The stents were then rinsed with deionized and distilled water, and immersed in an about 37% HCl acid bath at room temperature for about 7 minutes. Each stent was then rinsed with deionized and distilled water. After rinsing, each stent was immersed in an electrolytic bath containing nickel ions, which bath included NiCl dissolved in HCl and water. The nickel strike was conducted at room temperature. A negative electric charge was then applied to the stent causing the nickel ions to aggregate on the stent surface. A charge of approximately 0.7 volts and about 0.09 amps was applied for about 4 minutes. Subsequent to this electroplating step, the stents were again rinsed in distilled, deionized water. Following the electrolytic strike, the stents were immersed into an electroless Ni—P bath for about ten minutes, which bath included a mixture of NiSO₄, NaH₂PO₂, Na₃C₆H₅O₇, and NH₄Cl to form a homogenous, aqueous solution. The respective amounts of these materials in the bath formulation used were as follows (based upon 1 L preparation in deionized, distilled water): NiSO₄ (30 g/L); NaH₂PO₂ (10.6 g/L); Na₃C₆H₅₀₇ (100 g/L); and NH₄Cl (53.6 g/L). Adjustment of the pH was generally performed to target ranges by addition of NaOH.

Rapamycin (sirolimus), DM-1, and HP-1 were also added to various electroless Ni—P baths, of the same composition as the above described bath, and co-deposited therewith on the surface of the stent over the tie layer and initial Ni—P layer. The electroless Ni—P and drug co-depositions were conducted at a temperature range of between about 37-45 degrees C. and a pH of between about 9.5-10 for a total of about 120 minutes. DAA-1 and HP-1 were added to their respective Ni—P baths at a concentration of about 1.25 mg per 25 ml Ni—P solution. Rapamycin was added to its Ni—P bath at a concentration of about 1 mg per 25 ml of Ni—P solution.

Weight measurements were taken before and after coating according to the methods just described. Substantial weight gain is considered a measure of coating deposition. Stents coated in each of the drug baths noted above were observed to exhibit marked weight gain.

Drug elution testing in 37 degree aqueous baths was conducted. Elution of compounds into the test baths was confirmed over at least 48 hour time periods from stents coated with each of these drug-loaded baths according to the methods just described.

It is to be appreciated that a method of applying multiple thin metallic films to thereby assemble a metallic matrix incorporating one or more biologically active agents onto an implantable device has been herein disclosed, and provides benefit either with or without incorporating the bioerodable composite embodiments elsewhere herein described. Among other benefits, certain implantable medical devices, such as stents, are limited in their material choices due to the desire to have a passive surface for acceptable biocompatibility. For example, it is a reason why balloon-expandable stents have been fabricated from stainless steel, and, more recently, cobalt-chromium. It is therefore a further intended object and benefit of certain aspects of the coating depositions herein described to alleviate this material limitation by having a relatively passive coating encasing the stent. As such, the opportunities for chosen stent materials maybe expanded upon, and other properties such as strength and radiopacity need not be compromised by requiring substantially inactive surface properties of the scaffold material.

Although various aspects of the present invention have been described in accordance with the embodiments described, one of ordinary skill in the art will readily recognize that there could be variations to the embodiments and those variations would be within the spirit and scope of the present invention.

For example, as noted above, a biodegradable polymer or other form of top coat can be applied over the metallic composite matrix to delay or control release of the therapeutic agents from the matrix.

In addition, multiple layers containing different drugs can be applied by sequential immersion in multiple electroless electrochemical baths containing the different drugs. Moreover, materials such as barium or bismuth can be co-deposited in the electroless deposition step to increase the radiopacity of th implantable device.

And while devices such as joints and leads, for example, can be coated with drugs to lessen the inflammatory response, other implantable devices, such as detachable coils for treating and sealing off aneurysms, can be coated with agents to cause coagulation or a thrombogenic response. Moreover, other implants such as orthopedic implants, valves, filters, or temporary devices such as guidewires may be beneficially formed or treated according to the various embodiments herein described. Such medical device implants will generally be provided pre-packaged in a sterile environment container. Moreover, they may be provided individually or sold or packaged together with other devices, or provided with instructions for use in combination with such other devices, so as to form overall combination assemblies or systems intended for particular indicated medical uses. Such devices that are specifically surface treated according to the present aspects, modes, or embodiments are considered individually benefited by the present invention. Moreover, such overall combination assemblies and systems are further considered benefited and are thus contemplated as further aspects of the invention, though each component may not directly receive or provide the beneficial surface treatments herein described.

With regard to specific compounds that can be co-deposited in accordance with the invention, and with particular regard to stents, it is essentially limitless and includes for example; sirolimus or Rapamycin™, paclitaxel or Taxol™, growth factors, heparin, aspirin, tetracycline, dexamethasone, des-aspartate angiotensin I, tachykinins, sialokinins, apocynin, siRNA, pleiotrophin, exochelin, nitric oxide or nitric oxide donors, steroids, anti-inflammatories, anti-proliferatives, immunosuppressants, combinations or blends thereof, or analogs, precursors, or derivatives thereof.

More over, various particular bioactive agents ar herein described as examples for incorporation into the various aspects, modes, and embodiments herein described. It is to be appreciated that various combinations or blends thereof are also contemplated, and furthermore that analogs, derivatives, or precursor materials thereof are also contemplated. For example, certain generally non-functional molecular components or groups may be modified, added, or removed while substantially retaining a particular desired bioactivity of such noted compounds. Or, such compounds may be “complexed” with other molecules or groups that erode or are otherwise metabolized or separated in the body (e.g. enzymatic cleaving) so as to release the desired bioactive compound. Furthermore, certain materials, such as for example genetic material or other active agents, may be delivered that promote the in situ production or release of other such desired bioactive materials. For example, various agents are known to provide bioactivity at least in part by stimulating the local production and/or release of nitric oxide. Such many variations or modifications are contemplated within, and thus do not depart from, the intended broad scope of the various broad aspects and objects noted herein.

In addition, any of the aforementioned compounds could be clad with a biodegradable coating prior to mixing in the electroless bath for time release after diffusion from the metallic composite coating.

Various aspects, modes, and embodiments heretofore described above note use of certain bioerodable and/or biodegradable materials in certain composite materials and related assemblies and methods. In general with respect to the such uses, these terms are considered generally interchangeable unless specifically indicated otherwise.

It is contemplated that the incorporation of bioerodable materials within other porous coating structures provide various intended benefits, though such should not be considered limiting to the scope of the invention. For example, particle co-deposition is enhanced with electroless electrochemical methods by use of such composite micro- or nano-particles. In another regard, because such bioerodable composite material is held within micro or nano-pores at the surface, such composite material is not thus required to provide the structural integrity as a robust surface coating or structural component as is typically required by other conventional bioerodable approaches to drug delivery, in particular from stents or stent coatings. As such, it is considered a further benefit to allow for more drug loading, and less polymer, than such conventional approaches. For example, with respect to electroless electrochemical deposition, the required amount of polymer component in the composite is limited only to that amount as is required to maintain particle integrity and prevent substantial dissolving during the deposition bath process. It is considered therefore that much more drug, and less polymer, may be used in such applications versus other prior disclosures, thus reducing polymer burdens into tissues and improving biocompatibility and inflammation response.

With further respect to such bioerodable material aspects of the invention, many different such materials and particulate forms thereof may be suitable for use as herein described. However, certain particular formulations may be of particular benefit for certain applications. For example, in the circumstance of electroless electrochemical co-deposition of such particles, such coating environments will typically be either acidic, e.g. pH of less than about 6.0, or basic, e.g. pH of more than about 8.0 or 8.5. However, the intended environment for drug release within a patient's body is generally pH neutral, or ranging between about 6.5 to about 7.5. Accordingly, a formulation of bioerodable composite particles that maintain robust integrity at either the basic or acidic pH levels of typical electroless electrochemical co-deposition, but that are erodable at the more central pH ranges typical of the body environment, may be of particular benefit for such embodiments.

Nonetheless, where such pH compatibility is not found for a particular material choice, modified coating parameters may ensure desired results, such as for example adjusting pH and/or temperature and/or time, or other bath constituent materials, to enhance deposition in a manner minimizing erosion in the bath. For example, a coating environment that deposits the required amount of particles for a particular application within a time period that is less than the time constant for particle erosion in the bath may suffice.

In a further regard, certain bioactive agents are believed to denature when deposited according to the high alkalinity (or conversely low acidity) environments of certain electroless electrochemical coating solutions. The ability to capture such drugs in particulate form with another carrier compound, such as bioerodable compound, is a further benefit to protect the drugs during such deposition procedures.

Although the description above contains many specificities, these should not be construed as limiting the scope of the invention but as merely providing illustrations of some of the presently preferred embodiments of this invention. Thus the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. An endolumenal stent system, comprising:
an endolumenal stent;
a porous surface on the endolumenal stent and having a plurality of pores; and
a composite material located within each of the pores and comprising a bioerodable material in combination with a bioactive agent.

2. The system of claim 1, wherein the composite material comprises a plurality of particles.

3. The system of claim 2, wherein the particles comprise an outer diameter that is less than about 5 microns.

4. The system of claim 2, wherein the particles comprise an outer diameter that is less than about 2 microns.

5. The system of claim 2, wherein the particles comprise an outer diameter that is less than about 1 micron.

6. The system of claim 2, wherein the particles comprise a bioerodable polymer in combination with the bioactive agent.

7. The system of claim 2, wherein:
the particles comprise an outer diameter;
the pores comprise an inner diameter; and
the inner diameter is substantially equivalent to the outer diameter.

8. The system of claim 1, wherein the pores comprise an inner diameter that is less than about 5 microns.

9. The system of claim 1, wherein the pores comprise an inner diameter that is less than about 2 microns.

10. The system of claim 1, wherein the pores comprise an inner diameter that is less than about 1 micron.

11. The system of claim 1, wherein the porous outer surface comprises a material that is inherently porous.

12. The system of claim 1, wherein:
the porous outer surface comprises a material that is not inherently porous; and
the pores are formed within the material.

13. The system of claim 12, wherein the pores are laser cut into the material.

14. The system of claim 12, wherein the plurality of pores are photochemically etched into the material.

15. The system of claim 12, wherein the plurality of pores are chemically etched into the material.

16. The system of claim 1, wherein the porous outer surface comprises a sintered material.

17. The system of claim 1, wherein:
the endolumenal stent comprises a scaffold constructed from a first material;
the porous outer surface comprises a coating material located on the first material; and
the pores are located within the coating material.

18. The system of claim 17, wherein the coating material comprises a non-polymeric material.

19. The system of claim 18, wherein:
the non-polymeric material comprises an electrochemically deposited material.

20. The system of claim 19, wherein the electrochemically deposited material comprises an electrolessly electrochemically deposited material.

21. The system of claim 20, wherein the electrolessly electrochemically deposited material comprises a composite material with a metal and a reducing agent of the metal.

22. The system of claim 21, wherein the metal comprises nickel.

23. The system of claim 22, wherein the reducing agent comprises phosphorous.

24. The system of claim 22, wherein the first material comprises a stainless steel alloy.

25. The system of claim 22, wherein the first material comprises a nickel-titanium alloy.

26. The system of claim 21, wherein the metal comprises cobalt.

27. The system of claim 26, wherein the reducing agent comprises phosphorous.

28. The system of claim 26, wherein the first material comprises a cobalt-chromium alloy.

29. The system of claim 17, further comprising a second material between the first material and the coating material.

30. The system of claim 29, wherein the second material comprises an electroplated metal.

31. The system of claim 30, wherein the electroplated metal comprises electroplated nickel.

32. The system of claim 29, further comprising a third material between the second material and the coating material.

33. The system of claim 32, wherein:
the second material comprises electroplated metal;
the third material comprises a first layer of an electrolessly electrochemically deposited composite material with a metal and a reducing agent of the metal; and
the coating material comprises a second layer of an electrolessly electrochemically deposited composite material with a metal and a reducing agent of the metal, and further comprises the composite material.

34. The system of claim 1, wherein the bioactive agent comprises an anti-rest nosis agent.

35. The system of claim 1, wherein the bioactive agent comprises an anti-inflammatory agent.

36. The system of claim 1, wherein the bioactive agent comprises an anti-proliferative agent.

37. The system of claim 1, wherein the bioactive agent comprises an anti-proliferative agent in combination with an anti-inflammatory agent.

38. The system of claim 1, wherein the bioactive agent comprises des-aspartate angiotensin 1.

39. The system of claim 1, wherein the bioactive agent comprises at least one of sirolimus, tacrolimus, everolimus, paclitaxel, a steroid, exochelin, dexamethasone, nitric oxide, apocynin, gamma-tocopherol, an antibody, a growth factor, a combination or blend thereof, or an analog, precursor or derivative thereof.

40. The system of claim 1, wherein the ratio of the bioactive material to the bioerodable material in the composite material is at least about 0.5:1.

41. The system of claim 1, wherein the ratio of the bioactive material to the bioerodable material in the composite material is at least about 1:1.

42. The system of claim 1, wherein the ratio of the bioactive material to the bioerodable material in the composite material is at least about 1.5:1.

43. The system of claim 1, wherein the bioerodable material comprises a bioerodable polymer material.

44. An endolumenal stent system, comprising:
an endolumenal stent;
a plurality of composite particles coupled to the endolumenal stent;
wherein the composite particles comprise a bioerodable material in combination with a bioactive agent; and
wherein the composite particles are adapted to release the bioactive agent from the endolumenal stent when the endolumenal stent is implanted within a body of a patient.

45. A system for depositing a bioactive coating onto a surface of an endolumenal stent, comprising:
a coating environment;
a plurality of metal ions within the coating environment;
a plurality of particles located within the coating environment and that each comprises a bioactive agent in combination with a carrier material; and
wherein the coating environment is adapted to co-deposit the metal ions with the particles onto the endolumenal stent surface to form a composite surface coating when the endolumenal stent is exposed to the coating environment and such that the co-deposited composite surface coating is adapted to elute the bioactive agent therefrom when the surface is exposed to a body of a patient.

46. A system for depositing a bioactive coating onto a surface of an endolumenal stent, comprising:
a coating environment with a coating material;
a plurality of composite particles located within the coating environment and that comprise a bioerodable material in combination with a bioactive agent;
wherein the coating environment is adapted to co-deposit the coating material with the composite particles onto the surface so as to form a composite surface coating that is adapted to release the bioactive agent when exposed to a body of a patient.
